(19) United States
(12) Patent Application Publication
(10) Pub. No.: US 2020/0087611 A1
(43) Pub. Date: Mar. 19, 2020

(54) MICROORGANISM TESTING METHOD AND APPARATUS FOR THE SAME

(71) Applicant: SATAKE CORPORATION, Chiyoda-ku, Tokyo (JP)

(72) Inventors: Jianjun HE, Chiyoda-ku, Tokyo (JP); Masanori MATSUDA, Chiyoda-ku, Tokyo (JP)

(73) Assignee: SATAKE CORPORATION, Chiyoda-ku, Tokyo (JP)

(21) Appl. No.: 16/467,671

(22) PCT Filed: Nov. 24, 2017
(86) PCT No.: PCT/JP2017/042274

§ 371 Date: Jun. 7, 2019

(30) Foreign Application Priority Data
Dec. 9, 2016 (JP) 2016-239302

(51) Int. Cl. C12M 1/34; G01N 21/78; G01N 21/64; G01N 33/18; C12Q 1/06

(52) U.S. Cl. CPC C12M 41/36; G01N 21/78; C12Q 1/06; G01N 33/18; G01N 21/64

(57) ABSTRACT

A method and apparatus for detecting microorganisms in ballast water, the apparatus including: an excitation light source provided with light sources for emitting excitation light to irradiate an irradiated surface of sample solution continuously; photodetector detecting light of fluorescence emission caused by excitation light from the excitation light source control means converting the light detected by the photodetector to an electrical signal to detect and count number of light emissions, and estimating the number of microorganisms included in a sample within the sample container from the number of light emissions; and an operation unit electrically connected to the control means. The excitation light source uses two different kinds of excitation light sources including a light source emitting light with a wavelength region causing phytoplankton to emit chlorophyll fluorescence and a light source emitting light with a wavelength region causing microorganisms stained by the fluorescent staining reagent to emit fluorescence.

### TECHNICAL FIELD

The present invention relates to a microorganism testing method and an apparatus for the method and, in particular, to a microorganism testing method suitable for detecting microorganisms such as living plankton included in ballast water or the like, and an apparatus for the method.

### BACKGROUND ART

A ship without cargo sails loaded with ballast water in order to stabilize the ship and discharges the ballast water in a sea area where cargo is loaded.

Ballast water is usually discharged in a sea area different from a sea area where the ballast water is loaded. Therefore, there is a possibility that microorganisms such as plankton and bacteria included in the ballast water are carried to an area other than an original habitat, and problems of destruction of an ecosystem and the like are caused.

In order to cope with such problems, international rules about regulations of ballast water were formulated, and “International Convention for the Control and Management of Ships' Ballast Water and Sediments (the Ballast Water Management Convention)” has been adopted.

In “Guidelines for ballast water sampling (G2)” related to the Ballast Water Management Convention, permissible populations of living organisms included in ballast water discharged from ships are stipulated by classifying the permissible populations based on minimum sizes of the organisms, for example, 10 organisms/m³ or fewer for organisms the minimum size of which is 50 μm or larger (hereinafter referred to as “L-size organisms”), 10 organisms/mL or fewer for organisms the minimum size of which is between 10 μm and 50 μm, including 10 μm and excluding 50 μm (hereinafter referred to as “S-size organisms) and the like in “the ballast water discharge standard (D-2)”.

As techniques for confirming whether the discharge standard is satisfied or not at the time of discharging the ballast water, a technique in which seawater is pumped through a flow cell to measure an image (for example, Patent Literature 1), an apparatus collecting seawater as sample water after flowing seawater pumped by a pump through filters with different openings, adding a staining reagent to the sample water and irradiating excitation light while stirring the sample water, detecting light of fluorescence emission caused by the excitation light and counting the number of light emissions, and calculating the number of microorganisms included in the sample water from the number of light emissions (for example, Patent Literature 2 and Patent Literature 3) and the like have been known up to now.

The apparatus described in Patent Literature 1 is provided with a staining portion staining organisms with live cells existing in a liquid specimen while flowing the liquid specimen, a concentrating portion concentrating the stained specimen so that concentration of the organisms is increased while flowing the stained specimen, an individual measuring portion acquiring image information about individuals including the organisms in the concentrated specimen, and a control means performing measurement of the organisms from the image information about the individuals outputted from the individual measuring portion.

Thereby, a process of staining organisms in specimen liquid, a process of concentrating the organisms in the liquid, a process of acquiring information about the organisms in the liquid and the like can be performed by a flow method. Therefore, in comparison with a technique of performing each process by batch, it is possible to significantly shorten or eliminate waiting time required until a part of the specimen for which one process has been finished proceeds to the next process. Thus, there is an advantage of acquiring stable information about life or death of organisms in the sense of preventing deterioration of a stained state during the waiting time.

In the above apparatus according to Patent Literature 1, however, seawater is pumped sequentially through various kinds of processes, and there is a problem that the apparatus is large-scaled, and the manufacturing cost increases. Moreover, though water is pumped sequentially through the various kinds of processes so that the waiting time is shortened, there is a problem that at least several hours are required to complete measurement.

Each of the apparatuses described in Patent Literature 2 and 3 is provided with: stirring/mixing means performing stirring/mixing of sample solution obtained by adding a sample and a fluorescent staining reagent into a batch-type, sample container formed of material transmitting light, an excitation light source provided with light sources irradiating excitation light to an irradiated surface of the sample container while the sample solution is being stirred by the stirring/mixing means, photodetector detecting light of fluorescence emission caused by the excitation light from the excitation light source, and control means converting the light detected by the photodetector to an electrical signal to detect the number of light emissions and calculating the number of microorganisms included in the sample within the sample container from the number of light emissions.

Thereby, after adding a sample and a fluorescent staining reagent into a batch-type sample container, stirring/mixing in the sample container is performed by the stirring/mixing means; excitation light is then caused to be incident to the irradiated surface of the sample container while the sample solution is being stirred, and; furthermore, fluorescence emission of microorganisms is received by the photodetector. Therefore, in comparison with the case of performing measurement with leaving sample solution standing without stirring, the microorganisms brightly emit light in an extremely short time, and it becomes possible to easily measure the number of microorganisms in ballast water in a short time. Because of the batch type, it becomes possible to downsize the apparatus, and there is an advantage that the manufacturing cost decreases.

In the above apparatuses described in Patent Literature 2 and 3, however, there is a problem that it is difficult to detect some phytoplankton. Especially as for some diatoms having siliceous (glassy) shells around cells, among algae, which are phytoplankton, a staining agent FDA (a fluorescent staining reagent FDA) is not easily taken in, and, therefore, the amount of fluorescence emission is small, and detection is difficult.

### CITATION LIST

### Patent Literature

[Patent Literature 1]

Japanese Patent Laid-Open No. 2009-85898
[Patent Literature 2]

Japanese Patent Laid-Open No. 2014-42463
[Patent Literature 3]

Japanese Patent Laid-Open No. 2014-55796

### SUMMARY OF INVENTION

### Technical Problem

In view of the above problems, the technical subject of the present invention is to provide a method for detecting microorganisms in ballast water, the method capable of easily detecting such phytoplankton that a fluorescent staining reagent is not easily taken in, in a short time, and an apparatus for the method.

### Solution to Problem

In order to solve the above subject, a microorganism testing apparatus according to the present invention is an apparatus for measuring the number of microorganisms in sample solution, the microorganism testing apparatus being provided with: stirring/mixing for stirring/mixing the sample solution prepared by adding a sample and a fluorescent staining reagent into a batch-type sample container formed of light transmitting material and performing stirring/mixing of the sample solution; an excitation light source provided with light sources for emitting excitation light to irradiate an irradiated surface of the sample container continuously while the sample solution is being stirred by the starring/mixing means; photodetector for detecting light of fluorescence emission caused by the excitation light from the excitation light source; control means converting the light detected by the photodetector to an electrical signal to detect and count the number of light emissions, and estimating the number of microorganisms included in the sample within the sample container from the number of light emissions; and an operation unit electrically connected to the control means; wherein such technical means is taken that the excitation light source uses two different kinds of excitation light sources including the excitation light sources being a light source emitting light with a wavelength region causing phytoplankton to emit chlorophyll fluorescence and a light source emitting light with a wavelength region causing microorganisms stained by the fluorescent staining reagent to emit fluorescence.

According to the microorganism testing apparatus of the present invention, since the two different kinds of excitation light sources, i.e. the light source emitting the light with the wavelength region causing phytoplankton to emit chlorophyll fluorescence and the light source emitting the light with the wavelength region causing microorganisms stained by the fluorescent staining reagent to emit fluorescence are used for the excitation light source, it becomes possible to detect such phytoplankton that the fluorescent staining reagent is not easily taken in, by using the light source emitting the light with the wavelength region causing chlorophyll fluorescence emission, and, thereby, it becomes possible to easily detect both of phytoplankton and zooplankton without any omission in a short time.

In the microorganism testing apparatus, the excitation light source is arranged such that excitation light emitted therefrom is incident orthogonally to the irradiated surface of the sample container, and a light receiving surface of the photodetector is arranged such that fluorescence emission is received at an angle orthogonal to the excitation light of the excitation light source.

According to the microorganism testing apparatus, the excitation light source is arranged such that the excitation light emitted therefrom is incident orthogonally to the irradiated surface of the sample container, and the light receiving surface of the photodetector is arranged such that fluorescence emission is received at the angle orthogonal to the excitation light of the excitation light source. Therefore, the excitation light from the excitation light source is not incident directly to the light receiving surface of the photodetector, and difference in the amount of light between a background and fluorescence emission of microorganisms becomes extremely clear. Thus, the microorganism detection accuracy is improved.

In the microorganism testing apparatus, the control means is provided with an operation unit calculating a permissible number of microorganisms N with respect to a ballast water discharge criterion after determining each of a microbial population n1 acquired by chlorophyll fluorescence emission, a microbial population n2 acquired by fluorescence emission by the fluorescent staining reagent and a microbial population n3 acquired by both of the chlorophyll fluorescence emission and the fluorescence emission by the fluorescent staining reagent.

According to the above microorganism testing apparatus, the control means estimates the microbial population n3 as the permissible microbial population N for a complemented number after determining each of the microbial population n1 acquired by chlorophyll fluorescence emission, the microbial population n2 acquired by fluorescence emission by the fluorescent staining reagent and the microbial population n3 acquired by both of the chlorophyll fluorescence emission and the fluorescence emission by the fluorescent staining reagent. The permissible population N makes it possible to appropriately evaluate the number of microorganisms and evaluate and apply the ballast water discharge standards (D-2) the same as true evaluation.

A microorganism testing method according to the present invention is that for measuring the number of microorganisms in sample solution, the microorganism testing method including: a stirring/mixing process of performing stirring/mixing of sample solution obtained by adding a fluorescent staining reagent to a sample in a batch-type sample container; an excitation process for continuously irradiating excitation light to an irradiated surface of the sample container continuously while stirring the sample solution; a photodetection process for counting fluorescences of microorganisms caused to emit fluorescence by the excitation process; and a number-of-microorganisms estimating process for calculating the number of microorganisms included in the sample within the sample container from the number of light emissions detected by the photodetection process; wherein the excitation process causes phytoplankton to be excited by a light source emitting light with a wavelength region causing chlorophyll fluorescence emission, and causes microorganisms stained by the fluorescent staining reagent to be excited by a light source emitting light with a wavelength region causing fluorescence emission.

In the microorganism testing method, the number-of-microorganisms estimating process calculates a permissible number of microorganisms N with respect to a ballast water discharge criterion after determining each of a microbial population n1 acquired by chlorophyll fluorescence emission, a microbial population n2 acquired by fluorescence emission by the fluorescent staining reagent and a microbial population n3 acquired by both of the chlorophyll fluorescence emission and the fluorescence emission by the fluorescent staining reagent.

According to the above microorganism testing method, a method for detecting such phytoplankton that a fluorescent staining reagent is not easily taken in by using the light source emitting light with the wavelength region causing chlorophyll fluorescence emission is realized. Thereby, it is possible to easily detect both of phytoplankton and zooplankton without any omission in a short time.

In the microorganism testing method, the microorganism estimating process calculates a population of zooplankton by subtracting the microbial population n2 acquired by the fluorescence emission by the fluorescent staining reagent from the microbial population n3 acquired by both of the chlorophyll fluorescence emission and the fluorescence emission by the fluorescent staining reagent.

According to the microorganism testing method, it becomes possible to, by subtracting the microbial population n2 acquired by the fluorescence emission by the fluorescent staining reagent from the microbial population n3 acquired by both of the chlorophyll fluorescence emission and the fluorescence emission by the fluorescent staining reagent, calculate and count only the population of zooplankton by the microorganism estimating process.

### Advantageous Effects of Invention

According to the present invention, it is possible to provide a method for detecting microorganisms in ballast water, the method capable of easily detecting such phytoplankton that a fluorescent staining reagent is not easily taken in, in a short time, and an apparatus for the method.

### BRIEF DESCRIPTION OF DRAWINGS

FIG. 1 is a perspective view showing a whole microorganism testing apparatus of the present invention.

FIG. 2 is a schematic cross-sectional plan view of a measuring portion of the microorganism testing apparatus.

FIG. 3 is a block diagram showing an overall configuration of the measuring portion.

FIG. 4 is a schematic cross-sectional plan view of a measuring portion capable of easily detecting such phytoplankton that a fluorescent staining reagent is not easily taken in.

FIG. 5A is a schematic cross-sectional view showing one embodiment of collimator.

FIG. 5B is a schematic cross-sectional view showing another example of the one embodiment of the collimator.

FIG. 6A is a diagram showing a field of view obtained without a slit.

FIG. 6B is a diagram showing the field of view narrowed with a slit.

FIG. 7 is a flowchart showing a measurement flow of the microorganism testing apparatus of one embodiment of the present invention.

FIG. 8A is a Venn diagram of sets related to microbial populations n1 and n2.

FIG. 8B is a Venn diagram of a set related to a microbial population n3 at the time of simultaneously radiating the two kinds of LED light sources.

FIG. 8C is a Venn diagram of a set obtained by subtracting the microbial population n2 detected by the fluorescent staining reagent from the microbial population n3 at the time of simultaneously radiating the two kinds of LED light sources.

FIG. 9 is a schematic plan view showing a modification 1 of the measuring portion of FIG. 4.

FIG. 10 is a schematic plan view showing a modification 2 of the measuring portion of FIG. 4.

FIG. 11 is a schematic plan view showing a third modification 3 of the measuring portion of FIG. 4.

FIG. 12 is a graph showing a test of whether such phytoplankton that the fluorescent staining reagent is not easily taken in can be detected or not.

FIG. 13 is a graph showing the second test of the above.

FIG. 14 is an explanatory diagram when received signals of photodetectors 9A and 9B of the modification 2 are compared.

### DESCRIPTION OF EMBODIMENT

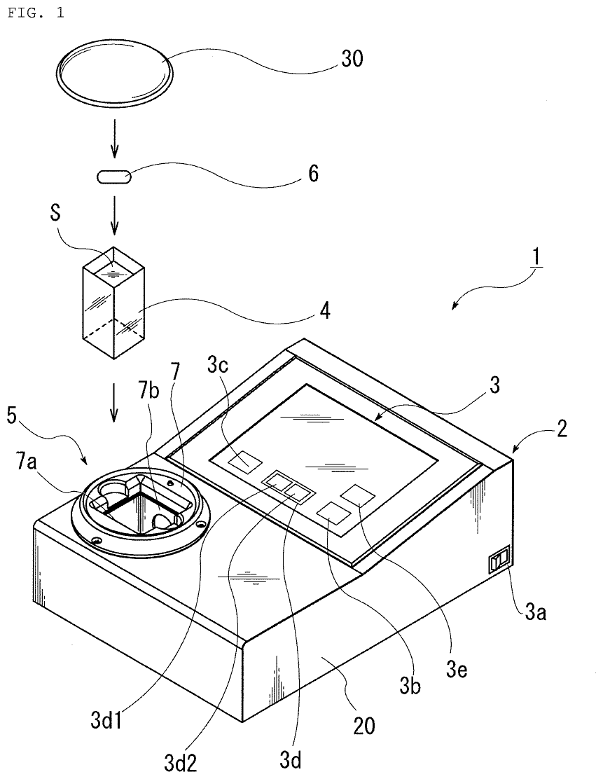

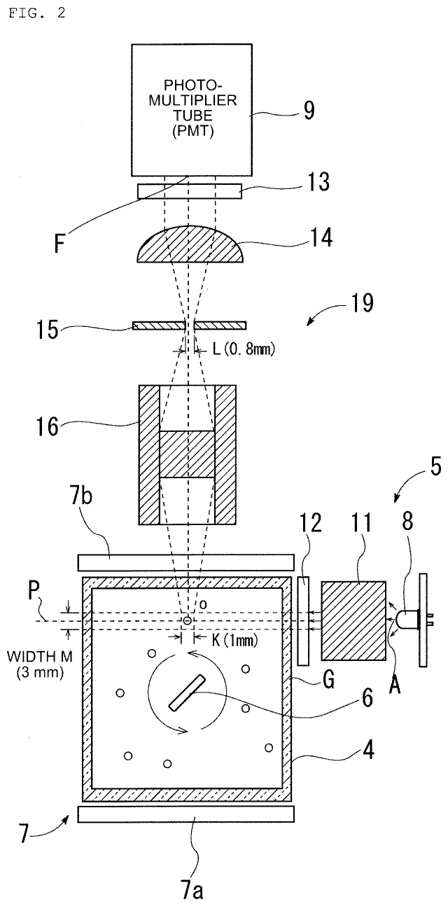

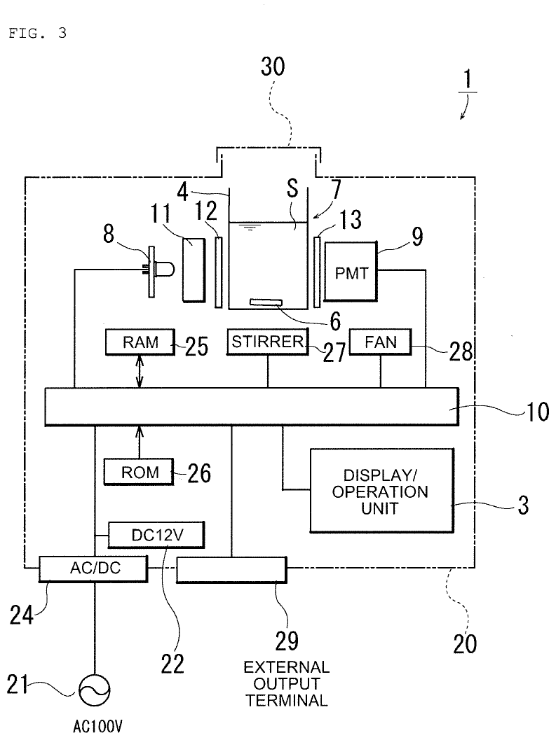

An embodiment for practicing the present invention will be described with reference to drawings. FIG. 1 is a perspective view showing a whole microorganism testing apparatus of the present invention; FIG. 2 is a schematic cross-sectional plan view of a measuring portion of the microorganism testing apparatus; and FIG. 3 is a block diagram showing an overall configuration of the measuring portion.

As shown in FIGS. 1, 2 and 3, a testing apparatus 1 of the present invention is configured with a body portion 2 that includes a control apparatus such as a CPU board and performs information processing work, statistical processing work and the like for measurement results and the like, a display/operation unit 3 on which operation icons and the like corresponding to operation buttons and the like that respond to a touch on a screen by a finger are arranged, and which is formed by a liquid crystal touch panel for displaying the measurement results and the like, and a measuring portion 5 that accommodates a batch-type sample container 4 formed of transparent material that transmits light (for example, glass, quarts, acrylic resin or the like) and that optically counts the number of microorganisms in sample solution S, the display/operation unit 3 and the measuring portion 5 being provided on the body portion 2 in line, as main portions.

Reference numeral 6 indicates a stirrer bar for stirring the sample solution S accommodated in the sample container 4. In the sample container 4, a sample, a luminescent reagent (a combination of the sample and the luminescent reagent is assumed to be the sample solution S) and the stirrer bar 6 are accommodated. A configuration is provided in which, when the sample container 4 is accommodated in the measuring portion 5, the stirrer bar 6 is driven and rotated by a magnetic stirrer built in the measuring portion 5. Thereby, it is possible to count the number of microorganisms in the sample solution S while stirring and mixing the sample solution S in the sample container 4 at a predetermined temperature. That is, in comparison with the case of counting the number of microorganisms in the sample solution S left standing, microorganisms brightly emit light in an extremely short time, and it becomes possible to easily measure the number of microorganisms in ballast water in a short time.

Dimensions of the testing apparatus 1 shown in FIG. 1 are: width-300 mm, depth=350 mm and height=130 mm. The weight is within a range of about 2 to 5 kg. The testing apparatus 1 can be accommodated in a handheld suitcase, a rucksack (also referred to as “a backpack”) (neither of them is shown) or the like and can be carried anywhere. The testing apparatus 1 is designed to be also driven by an AC power source or a battery so that measurement in a ship and outdoors is possible.

The sample container 4 is formed of transparent material that transmits light and is formed in a prismatic shape with a bottom face of 50 mm×50 mm and a height of 60 mm. The amount of content when a water level is 40 mm is set to 100 ml (milliliters). The sample container 4 is not limited to such a prismatic shape but may be in a cylindrical shape or a cubic shape if the amount of content of about 100 ml (milliliters) can be secured.

As shown in FIGS. 1, 2 and 3, the measuring portion 5 is provided with a sample container accommodating portion 7 that accommodates and holds the sample container 4, a light source portion 8 that radiates excitation light toward the sample container 4, and a photodetector 9 for observing microorganisms drifting in the sample container 4 through the excitation light irradiated from the light source portion 8. From the photodetector 9, communication is electrically performed to a CPU board 10 that counts the number of microorganisms in the sample solution S and performs information processing work, statistical processing work and the like of a measurement result and the like.

The sample container accommodating portion 7 is formed by holding plates 7a and 7b surrounding at least two faces of the sample container 4, and accommodates and holds the sample container 4 such that radiation of light from the light source portion 8 is not blocked.

As shown in FIG. 2, the light source portion 8 is arranged such that excitation light along a normal line AP is incident to an irradiated surface G of the sample container 4. The light source portion 8 is provided with an LED light source 8 arranged near the sample container accommodating portion 7, collimator 11 arranged on the front of the LED light source 8, the collimator 11 converting diffused light to parallel light (since an LED device emits light that is diffusedly radiated in random directions from a device side, the light is to be converted to parallel light to apply light beams uniformly to one surface at the same angle), and a band pass filter for excitation light 12 that causes excitation light constituted by the parallel light to be radiated to the sample container 4.

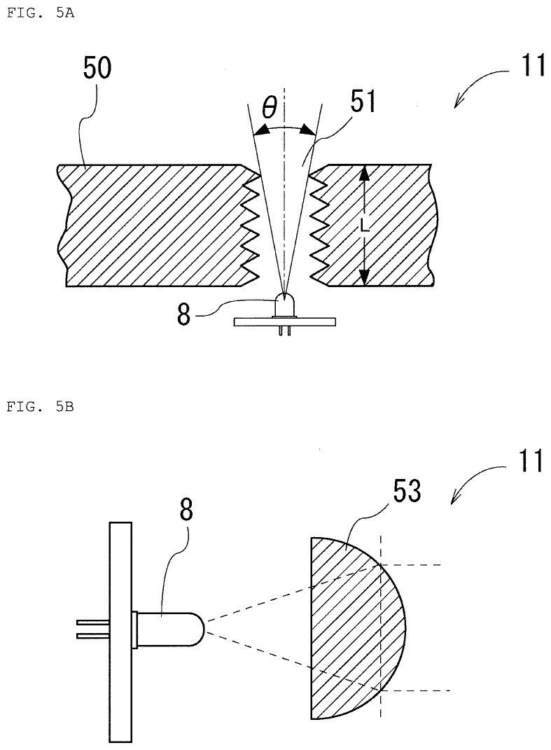

FIG. 5 shows schematic cross-sectional views showing one embodiment of the collimator 11. An example shown in FIG. 5A is an example in which the collimator 11 is formed by drilling a threaded hole 51 of a predetermined diameter in a flat plate 50 of a predetermined thickness, and a thickness L of the flat plate 50 and a hole diameter of the threaded hole 51 are appropriately set according to an optical path length. Thereby, diffused light at an incident angle θ that is irradiated from the LED light source 8 is converted to parallel light when passing through the threaded hole 51. In an example shown in FIG. 5B, an optimal condition between θ and L is decided by an S/N ratio test. For example, when M3 (screw hole outer diameter)×0.5 (pitch) is assumed, an optimal result is obtained when θ is 9.5°, and L is 15 mm.

The collimator 11 shown in FIG. 5B is provided with a convex lens 53 on the front of the LED light source 8, and diffused light radiated from the LED light source 8 is converted to parallel light when passing through the convex lens 53 to be emitted outside.

Though the light source portion 8 of the present embodiment uses an LED light source as a light source, the light source portion 8 is not limited to an LED light source, but a parallel light LED light source, a laser light source or a light bulb capable of radiating parallel light can be adopted if it is possible to cause fluorescent materials included in microorganisms to be excited. It goes without saying that, in the case of adopting a parallel light LED, a laser light source or a light bulb capable of radiating parallel light, the collimator 11 described above is unnecessary.

As shown in FIG. 2, the photodetector 9 is provided such that a light receiving surface F is arranged at an angle orthogonal to excitation light along the normal line AP from the light source portion 8. The photodetector 9 is also provided with a photomultiplier tube (PMT) 9 arranged and configured so that fluorescence is received along a light axis orthogonal to such parallel light that excitation light is radiated from the LED light source 8 toward the sample container 4, a band pass filter for fluorescence 13 arranged on the front of the photomultiplier tube (PMT) 9, a condenser lens 14 arranged on the front of the band pass filter for fluorescence 13, a slit 15 arranged on the front of the condenser 14 and a relay lens 16 installed in a gap between the slit 15 and the sample container 4, the relay lens 16 being for causing fluorescent materials included in microorganisms to excite and condensing fluorescence emitted thereby to form an image.

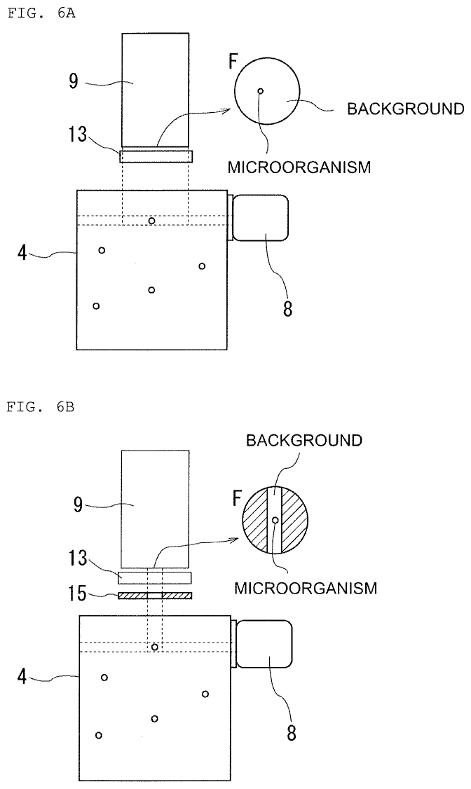

The slit 15 narrows a field of view to be in a slit shape. That is, as shown in FIG. 6, though such a background that the light receiving surface F is formed in a circle is monitored in a state without a slit in FIG. 6A, such a background that the light receiving surface F is formed by a rectangular slit excluding shaded parts is monitored in a state with a slit in FIG. 6B. Therefore, as a result of a monitoring area (a monitoring range) on the observation surface F being narrowed as in FIG. 6B, the area of fluorescence emission on the background, which is to be noise, is also narrowed. Thus, the ratio of a signal of fluorescence emission of microorganisms to fluorescence emission of the background is improved, and the accuracy of detection of the fluorescence emission of the microorganisms is improved.

Though an example has been shown in which the photodetector 9 uses a photomultiplier tube (PMT) as a photodetector, the photodetector is not limited to a photomultiplier tube (PMT), but various kinds of light detectors capable of detecting light emission of fluorescent materials included in microorganisms similarly to a photomultiplier tube (PMT), such as a silicon photodiode (SiPD) and an avalanche photodiode (APD), can be adopted.

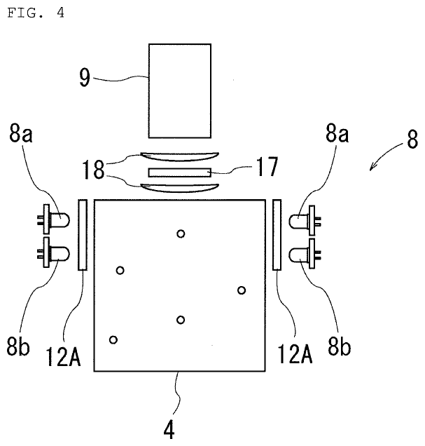

Next, description will be made on a configuration capable of easily detecting such phytoplankton that a fluorescent staining reagent is not easily taken in, the configuration being a main part of the present invention, with reference to FIG. 4.

The light source portion 8 shown in FIG. 4 is characterized in that two kinds of LED light sources 8a, 8b with different wavelength regions are used. That is, the LED light source 8 is provided with a pair of the LED light source 8a that emits bluish-green light around a wavelength region of 490 nm (a light source similar to a conventional one) and the LED light source 8b that emits bluish-purple light around a wavelength region of 450 nm. It is recommended to provide the pair of the LED light sources 8a, 8b in such a manner of facing each other sandwiching the sample container 4. Between the LED light sources 8a, 8b and the sample container 4, band pass filters for excitation light 12A, 12A that transmit light with a wavelength region of 395 to 505 nm are interposed, respectively, so that light on the LED light source 8b side is easily transmitted. These wavelength regions are merely an example and can be appropriately changed according to conditions.

A long pass filter 17 that transmits a light with a wavelength region of 510 nm or more is provided on the front of the photodetector 9 shown in FIG. 4. Furthermore, condenser lenses 18 are arranged in front of and behind the long pass filter 17 to sandwich the long pass filter 17.

Furthermore, an electrical control configuration will be described with reference to FIG. 3. In the center inside a case 20 forming the body portion 2, the CPU board 10 is arranged, the CPU board 10 receiving power source supply from an AC power source 21 or a secondary battery 22 to analyze an output signal converted from light to electricity by the photomultiplier tube (PMT) 9, judge whether or not brightness is within or above an arbitrary brightness range, count pulses of a signal with an arbitrary brightness and perform on/off control of the LED light source 8. An AC/DC converter 24 is interposed between the AC power source 21 and the CPU board 10.

Each of the photomultiplier tube (PMT) 9, the LED light source 8, a RAM 25 to be a storage portion for reading and writing and a ROM 26 to be a storage portion dedicated for reading is electrically connected to the CPU board 10. Further, they are electrically connected to the display/operation unit 3 formed by a liquid crystal touch panel or the like shown in FIG. 1. A configuration is provided in which on/off switching control is performed by pressing down a power source button 3a displayed on the liquid crystal panel, measurement is started by pressing down a measurement start button 3b, transfer of data to an external printer or personal computer is performed by pressing down an external output button 3c, switching between measurement types (switching between measurement of L-size microorganisms (3d1) or measurement of S-size microorganisms (3d2)) is performed by pressing down a setting button 3d, and change of setting of a judgment criterion, change of setting of a threshold and change of setting of measurement time can be performed by pressing down a menu button 3e, as described later.

In addition, a magnetic stirrer 27 that causes the stirrer bar 6 to rotate by magnetic force, a cooling fan 28 for control equipment, and external output terminals 29, such as RS-232C and universal serial bus (USB) terminals, are connected to the CPU board 10.

FIG. 7 is a flowchart showing a measurement flow. Operation in the above configuration will be described with reference to FIGS. 1 to 7.

### [Measurement of Chlorophyll Fluorescence]

First, measurement of chlorophyll fluorescence is started. A operator takes 100 ml (milliliters) of ballast water as a sample using a pipette or the like and injects the ballast water into the sample container 4 (step 1 in FIG. 7). Next, by accommodating the sample container 4 into the measuring portion 5 of the testing apparatus 1 and applying a cover 30 of the measuring portion 5, measurement preparation is completed.

The operator turns on the power source button 3a on the body portion 2 and makes preparations by pressing down the setting button 3d, the menu button 3e and the like on the display/operation unit 3 configured with a liquid crystal touch panel. After that, the measurement start button 3b is turned on. Thereby, the LED light sources 8b, 8b for chlorophyll fluorescence are lit up (see FIG. 4 and step 2 in FIG. 7), and light transmitted through the band pass filters for excitation light 12A, 12A (FIG. 4) is irradiated to the sample container 4. At this time, for example, light with a wavelength of 450 nm as a wavelength characteristic is irradiated, and chlorophyll components of a specimen (microorganisms) in the sample container 4 emit fluorescence. The fluorescence by the chlorophyll components is transmitted through the long pass filter 17 and detected by the photomultiplier tube (PMT) 9 (step 3 in FIG. 7).

In the photomultiplier tube (PMT) 9, light energy is converted to electrical energy by using a photoelectric effect, and a current amplifying function is added, so that fluorescence emission of the chlorophyll components with a high sensitivity. A detected electrical signal is sent to the CPU board 10, and received light waveforms at or above a predetermined threshold are counted (step 4 in FIG. 7).

Furthermore, in the CPU board 10, the number of microorganisms existing in the 100 ml (milliliters) of water, in the sample container 4 is estimated from the counted value of the received light waveforms, and the number of microorganisms is displayed on the display/operation unit 3 (step 5 in FIG. 7).

### EXAMPLE 1

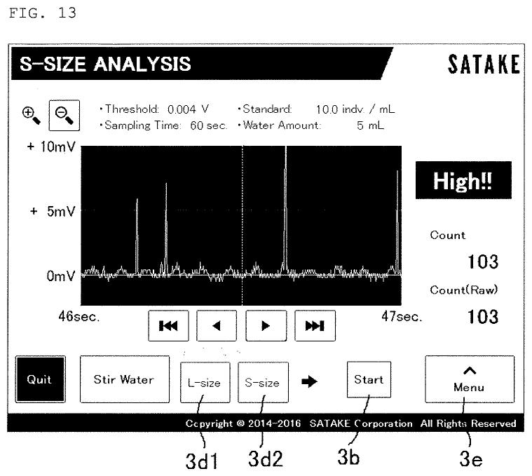

With Prorocentrum micans, which is a kind of phytoplankton, used as test microorganisms, it was verified whether or not the population can be estimated by the photomultiplier tube (PMT) 9 by chlorophyll fluorescence. A plurality of Prorocentrum micans individuals are accommodated in the sample container 4 (with a capacity of 100 mL) together with water, and the counted number of waveforms was detected (see FIGS. 12 and 13). As a result, from the obtained counted number of waveforms, a population of 102 was counted in FIG. 12, and a population of 103 was counted in FIG. 13. That is, it was known that, for phytoplankton existing in the 100 mL of ballast water, especially even for phytoplankton that does not easily absorb FDA and the like, a microbial population can be estimated by chlorophyll fluorescence emission without absorption of FDA. In the CPU board 10, the microbial population at this time is stored as n1 (step 5 in FIG. 7).

### [Measurement of Fluorescence by Staining Liquid]

Next, returning to FIG. 7, measurement of fluorescence by staining liquid will be described. The sample container 4 after the measurement of the chlorophyll fluorescence emission is taken out of the testing apparatus 1 (step 6 in FIG. 7), and a fluorescent staining reagent is added into the taken-out sample container 4 (step 7 in FIG. 7).

Commonly known Calcein-AM (manufactured by PromoCell GMBH in Germany), FDA or the like can be used as the fluorescent staining reagent. Calcein-AM tends to stain phytoplankton, while FDA tends to stain zooplankton. Then, by the operator causing the sample container 4 to be accommodated in the measuring portion 5 of the testing apparatus 1 after putting the stirrer bar 6 into the sample container 4 and applying the cover 30, measurement preparation is completed.

Here, the operator presses down an S size setting button 3d2 (or L size 3d1) on the display/operation unit 3 and turns on the measurement start button 3b. Then, the stirrer bar 6 rotates by driving of the magnetic stirrer 27 built in the measuring portion 5, and the sample solution S is stirred (step 8 in FIG. 7).

Next, the LED light sources 8a, 8a are lit up (see FIG. 4), and light transmitted through the band pass filters for excitation light 12A, 12A is irradiated to the sample container 4. At this time, for example, light around a wavelength region of 490 nm as a wavelength characteristic is irradiated, and the specimen (the microorganisms) in the sample container 4 emit fluorescence. Then, the fluorescence is transmitted through the band pass filter for fluorescence 15 and detected by the photomultiplier tube (PMT) 9 (step 10 in FIG. 7).

A detected electrical signal detected by the photomultiplier tube (PMT) 9 is sent to the CPU board 10, and received light waveforms at or above a predetermined threshold are counted (step 11 in FIG. 7). Furthermore, in the CPU board 10, the number of microorganisms existing in the 100 ml (milliliters) of water in the sample container 4 is estimated from the counted value of the received light waveforms, and the number of microorganisms is displayed on the display/operation unit 3. In the CPU board 10, the microbial population at this time is stored as n2 (step 12 in FIG. 7).

[Measurement of both of Chlorophyll Fluorescence and Fluorescence by Staining Liquid]

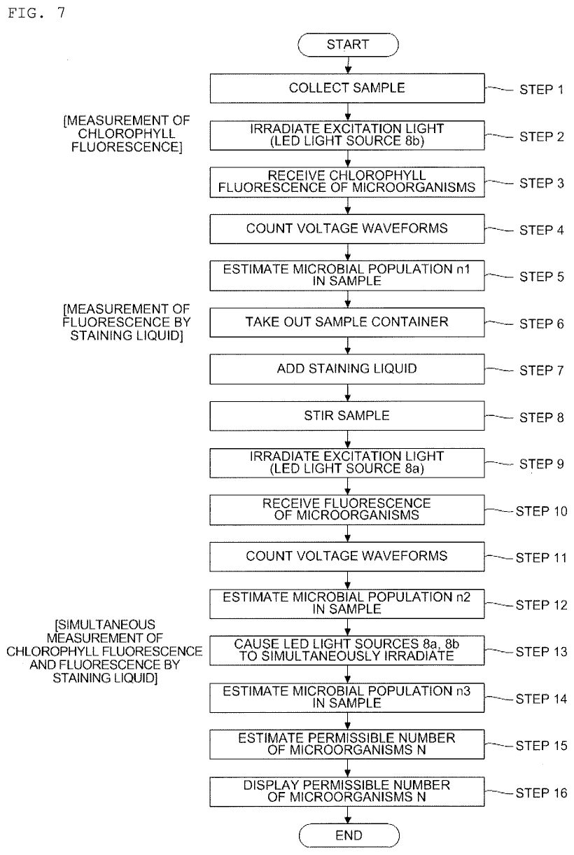

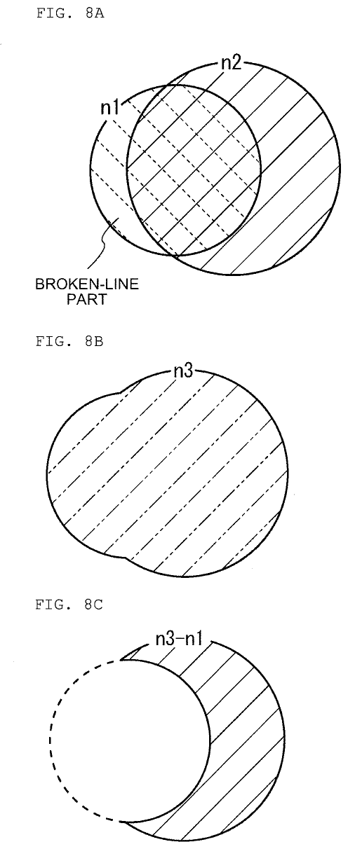

Then, both of the LED light sources 8a, 8a and the LED light sources 8b, 8b are caused to simultaneously radiate (step 13 in FIG. 7); detection is performed by the photomultiplier tube (PMT) 9; and the microbial population at this time is stored as n3 (step 14 in FIG. 7). Here, description will be made on a relationship among the microbial population n1 detected by the chlorophyll fluorescence emission, the microbial population n2 detected by the fluorescence emission using the fluorescent staining reagent and the microbial population n3 at the time of simultaneously radiating the two kinds of LED light sources 8a, 8b, using FIG. 8.

FIG. 8 shows Venn diagrams of sets related to the microbial populations n1, n2 and n3 acquired by the above process. FIG. 8A shows a logical sum set obtained by two sets of the microbial population n1 and the microbial population n2 being combined. Since only the microbial population n2 detected by fluorescence emission using a fluorescent staining reagent has been evaluated heretofore, there is a possibility that the population of phytoplankton that does not easily absorb the fluorescent staining reagent (a broken-line part of the microbial population n1), the phytoplankton not having been detected conventionally, has not been taken into account. This means that an allowable microbial population is estimated less than the true allowable microbial population, and the ballast water discharge standard (D-2) is also evaluated more loosely than true evaluation.

Therefore, if the microbial population at the time of simultaneously radiating the two kinds of LED light sources 8a, 8b is assumed as n3 as in FIG. 8B, the population of phytoplankton that does not easily absorb the fluorescent staining reagent is added, and an appropriate permissible microbial population can be obtained. The CPU board 10 estimates the microbial population n3 at the time of simultaneously radiating the two kinds of LED light sources 8a, 8b as a permissible microbial population N for a complemented number (step 15 in FIG. 7), and the permissible population N is displayed on the display/operation unit 3 based thereon (step 16 in FIG. 7). Since the permissible population N appropriately estimates the number of microorganisms, it is possible to evaluate and apply the ballast water discharge standards (D-2) the same as true evaluation.

FIG. 8C is a diagram in which the microbial population n2 detected by the fluorescent staining reagent is subtracted from the microbial population n3 at the time of simultaneously radiating the two kinds of LED light sources 8a, 8b. The set of n3-n2 is such that the set n2 of only phytoplankton is subtracted from the set n3 in which zooplankton and the phytoplankton coexist, and only the population of the zooplankton can be determined.

The [measurement of chlorophyll fluorescence] described in paragraph 0042 and the [measurement of fluorescence by staining liquid] described in paragraph 0047 may be exchanged in order and implemented. Further, the [measurement of both of chlorophyll fluorescence and fluorescence by staining liquid] described in paragraph 0052 may be implemented first.

As described above, according to the present embodiment, there is provided a microorganism testing apparatus provided with the body portion 2, the display/operation unit 3 and the measuring portion 5 optically counting the number of microorganisms in the sample solution S accommodated in the batch-type sample container 4, the display/operation unit 3 and the measuring portion 5 being arranged in line on the body portion 2, wherein

the measuring portion 5 is configured being provided with the sample container accommodating portion 7 accommodating and holding the sample container 4, the light source portion 8 radiating excitation light toward the sample container 4, and the photodetector 9 for observing microorganisms drifting in the sample container 4 by the excitation light radiated from the light source portion 8; and

the two different kinds of LED light sources 8a, 8b with different wavelength regions (especially, the LED light source 8a emitting bluish-green light around the wavelength region of 490 nm (a light source similar to a conventional one) and the LED light source 8b emitting bluish-purple light around the wavelength region of 450 nm are provided as a pair) are used for the light source portion 8. Therefore, by easily detecting such phytoplankton that a fluorescent staining reagent is not easily taken in, in a short time, it becomes possible to detect both of zooplankton and phytoplankton without failure.

### EXAMPLE 2

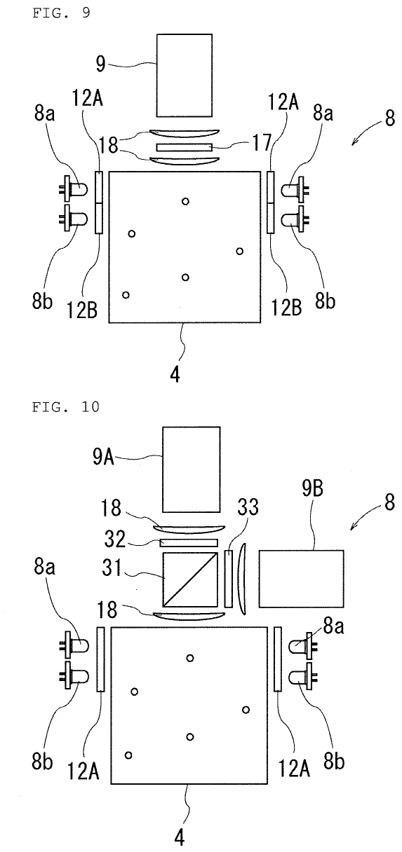

FIG. 9 shows a modification 1 of the measuring portion, the modification 1 being characterized in that the two kinds of LED light sources 8a, 8b are provided with dedicated band pass filters 12A, 12B, respectively, in comparison with a basic example of the measuring portion of FIG. 4.

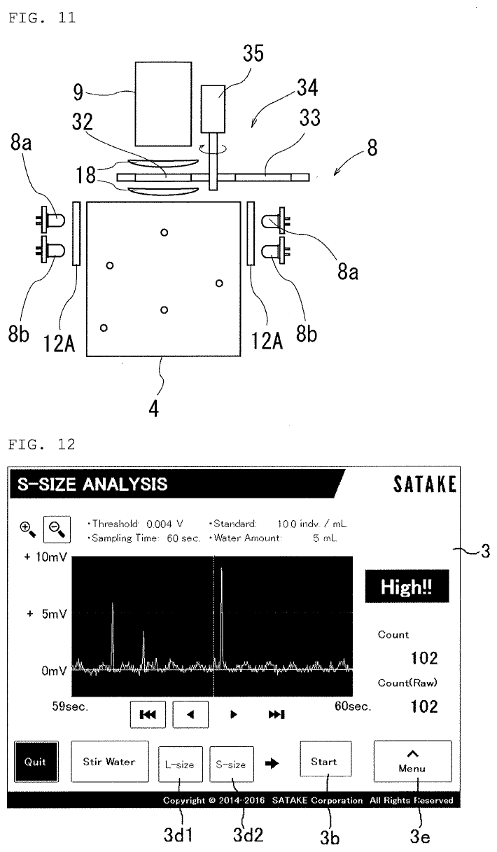

FIG. 10 shows a modification 2 of the measuring portion, the modification 2 being characterized in that a dichroic mirror 31 capable of spectroscopy and two photodetectors 9A, 9B with sensitivities specific to a wavelength obtained by spectroscopy are provided, in comparison with the basic example of the measuring portion of FIG. 4. A long pass filter 33 that transmits a wavelength region with a 650 nm or more is interposed between the photodetector 9A and the dichroic mirror 31, and a band pass filter 32 with a wavelength region of 510 to 550 nm is interposed between the photodetector 9A and the dichroic mirror 31.

FIG. 11 shows a modification 3 of the measuring portion, the modification 3 being characterized in that, instead of the single long pass filter 17, a filter wheel 34 configured with the band pass filter 32 with the wavelength region of 510 to 550 nm and the long pass filter 33 that transmits the wavelength region of 650 nm or more is arranged, in comparison with the basic example in FIG. 4. Reference numeral 35 in FIG. 11 indicates a step motor that drives the filter wheel 34.

In the modification 1 of the measuring portion shown in FIG. 9, the modification 2 of the measuring portion shown in FIG. 10 and the modification 3 of the measuring portion shown in FIG. 11, the light source portion 8 is provided with the two kinds of LED light sources 8a, 8b with different wavelength regions, and a filter and a photodetector specific to each of the light sources are provided. Therefore, by easily detecting such phytoplankton that a fluorescent staining reagent is not easily taken in, in a short time, both of zooplankton and phytoplankton can be detected without failure.

Further, as described before, the CPU board 10 determines each of the microbial population n1 acquired by chlorophyll fluorescence emission, the microbial population n2 acquired by fluorescence emission by staining liquid and the microbial population n3 acquired by both of the chlorophyll fluorescence emission and the fluorescence emission by the staining liquid.

The microbial population n3 is such that the population of phytoplankton that does not easily absorb a fluorescent staining reagent is added, and can be an appropriate permissible microbial population.

The CPU board 10 estimates a permissible microbial population N for the complemented number of microorganism. Since the permissible population N appropriately estimates the number of microorganisms, such operation/effects that it is possible to evaluate and apply the ballast water discharge standards (D-2) the same as true evaluation.

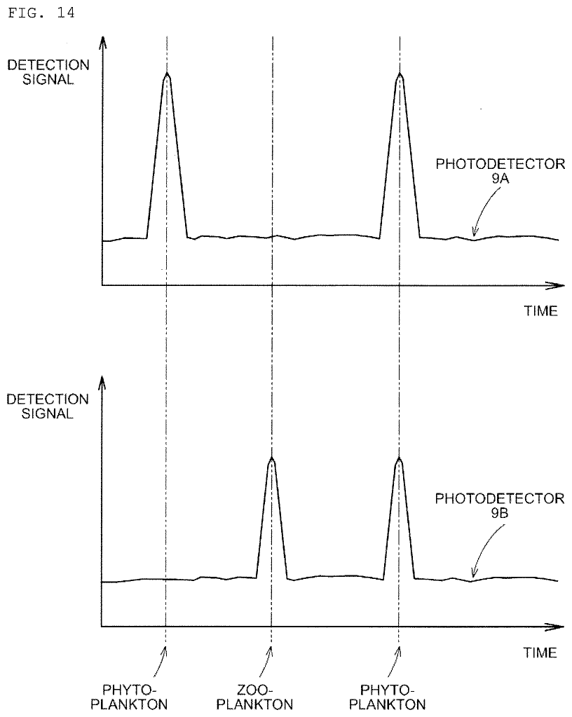

In the case of the modification 2 in FIG. 10, while the photodetector 9A mainly detects fluorescence emission only of phytoplankton, the photodetector 9B can detect fluorescence emission of both of the phytoplankton and zooplankton. As shown in FIG. 14, by comparing the photodetector 9A and the photodetector 9B, it is possible to, if there is a signal that cannot be detected by the photodetector 9A but detected by the photodetector 9B, estimate the signal as zooplankton. By counting the number of such signals, it becomes possible to grasp the population only of zooplankton.

### INDUSTRIAL APPLICABILITY

The present invention can be applied to a microorganism testing apparatus for confirming whether ballast water satisfies a discharge criterion at the time of discharging the ballast water.

### REFERENCE SIGNS LIST

1 testing apparatus
2 body portion
3 display/operation unit
4 sample container
5 measuring portion
6 stirrer bar
7 sample container accommodating portion
8 light source portion
9 photodetector
10 CPU board
11 collimator
12 band pass filter for excitation light
13 band pass filter for fluorescence
14 condenser lens
15 slit
16 relay lens
17 long pass filter
18 condenser lens
20 case
21 AC power source
22 secondary battery
24 AC/DC converter
25 RAM
26 ROM
27 magnetic stirrer
28 cooling fan
29 external output terminal
30 cover
31 dichroic mirror
32 band pass filter
33 long pass filter
34 filter wheel
35 step motor
50 flat plate
51 threaded hole
53 convex lens

1. A microorganism testing apparatus for measuring the number of microorganisms in sample solution, the microorganism testing apparatus comprising:
stirring/mixing unit adding a sample and a fluorescent staining reagent into a batch-type sample container formed of material transmitting light and performing stirring/mixing of the sample solution;
an excitation light source provided with light sources for emitting excitation light to irradiate to an irradiated surface of the sample container continuously while the sample solution is being stirred by the starring/mixing unit;
photodetector for detecting light of fluorescence emission caused by the excitation light from the excitation light source;
control means converting the light detected by the photodetector to an electrical signal to detect and count the number of light emissions, and calculating the number of microorganisms included in the sample within the sample container from the number of light emissions; and
an operation unit electrically connected to the control unit; wherein the excitation light source uses two different kinds of excitation light sources, the excitation light sources being a light source emitting light with a wavelength region causing phytoplankton to emit chlorophyll fluorescence and a light source emitting light with a wavelength region causing microorganisms stained by the fluorescent staining reagent to emit fluorescence.

2. The microorganism testing apparatus according to claim 1, wherein the excitation light source is arranged such that excitation light emitted therefrom is caused to be incident orthogonally to the irradiated surface of the sample container, and a light receiving surface of the photodetector is arranged such that fluorescence emission is received at an angle orthogonal to the excitation light of the excitation light source.

3. The microorganism testing apparatus according to claim 1, wherein the control unit comprises an operation unit calculating a permissible number of microorganisms N with respect to a ballast water discharge criterion after determining each of a microbial population n1 acquired by chlorophyll fluorescence emission, a microbial population n2 acquired by fluorescence emission by the fluorescent staining reagent and a microbial population n3 acquired by both of the chlorophyll fluorescence emission and the fluorescence emission by the fluorescent staining reagent.

4. A microorganism testing method for measuring the number of microorganisms in sample solution, the microorganism testing method comprising:
a stirring/mixing process of performing stirring/mixing of sample solution obtained by adding a fluorescent staining reagent to a sample in a batch-type sample container;
an excitation process for irradiating excitation light to an irradiated surface of the sample container continuously while stirring the sample solution;
a photodetection process of counting fluorescences of microorganisms caused to emit fluorescence by the excitation process; and
a number-of-microorganisms estimating process of calculating the number of microorganisms included in the sample within the sample container from the number of light emissions detected by the photodetection process; wherein the excitation process causes phytoplankton to be excited by a light source emitting light with a wavelength region causing chlorophyll fluorescence emission and causes microorganisms stained by the fluorescent staining reagent to be excited by a light source emitting light with a wavelength region causing fluorescence emission.

5. The microorganism testing method according to claim 4, wherein the number-of-microorganisms estimating process comprises calculating a permissible number of microorganisms N with respect to a ballast water discharge criterion after determining each of a microbial population n1 acquired by chlorophyll fluorescence emission, a microbial population n2 acquired by fluorescence emission by the fluorescent staining reagent and a microbial population n3 acquired by both of the chlorophyll fluorescence emission and the fluorescence emission by the fluorescent staining reagent.

6. The microorganism testing method according to claim 5, wherein the microorganism estimating process comprises calculating a population of zooplankton by subtracting the microbial population n2 acquired by the fluorescence emission by the fluorescent staining reagent from the microbial population n3 acquired by both of the chlorophyll fluorescence emission and the fluorescence emission by the fluorescent staining reagent.

7. The microorganism testing apparatus according to claim 2, wherein the control unit comprises an operation unit calculating a permissible number of microorganisms N with respect to a ballast water discharge criterion after determining each of a microbial population n1 acquired by chlorophyll fluorescence emission, a microbial population n2 acquired by fluorescence emission by the fluorescent staining reagent and a microbial population n3 acquired by both of the chlorophyll fluorescence emission and the fluorescence emission by the fluorescent staining reagent.
